(19) United States
(12) Patent Application Publication
(10) Pub. No.: US 2009/0312629 A1
(43) Pub. Date: Dec. 17, 2009

(54) CORRECTION OF RELATIVE TRACKING ERRORS BASED ON A FIDUCIAL

(75) Inventors: Sharif Razzaque, Chapel Hill, NC (US); Andrei State, Chapel Hill, NC (US); Caroline Green, Chapel Hill, NC (US); Kurtis Keller, Hillsborough, NC (US)

Correspondence Address: KNOBBE MARTENS OLSON & BEAR LLP, 2040 MAIN STREET, FOURTEENTH FLOOR, IRVINE, CA, 92614, US

(73) Assignee: INNEROPTIC TECHNOLOGY INC., Hillsborough, NC (US)

(21) Appl. No.: 12/483,099

(22) Filed: Jun. 11, 2009

Related U.S. Application Data
(60) Provisional application No. 61/131,840, filed on Jun. 13, 2008.

(51) Int. Cl. A61B 5/05; A61B 8/00

(52) U.S. Cl. 600/426; 600/437

(57) ABSTRACT

Presented herein are methods, systems, devices, and computer-readable media for correction of relative tracking error based on a fiducial. One embodiment is a method for the correction of obtained emplacement data of two surgical instruments by the detection of a detectable fiducial coupled to first surgical instrument by a second surgical instrument or something thereto coupled. The corrected relative emplacements of both surgical instruments is then determined based on the emplacement of the fiducial. This corrected emplacement is then used to produce an image for display. Systems and devices for carrying out the presented methods are also described.

### CROSS-REFERENCE TO RELATED APPLICATION

This application claims the benefit of U.S. Provisional Application No. 61/131,840, filed Jun. 13, 2008.

### BACKGROUND

In conventional minimally invasive surgery (MIS), the surgeon operates through small incisions using special instruments while viewing internal anatomy and the operating field on a video monitor. This is enabled through use of a camera such as an endoscope (e.g., a camera mounted in a tube suitable for insertion into the body). In order to make MIS easier and faster for the surgeon, and safer for the patient, a system may employ a tracker, which measures the position and orientation of the endoscope and other surgical instruments, so that computer graphics imagery can be generated to give the surgeon more information about the spatial relationships among the tracked instruments. For example, a system could superimpose a line over the live video from the endoscope's camera, to indicate the forward trajectory of a needle. Such as is described in U.S. patent application Ser. No. 12/399,899 (“the '899 application”), filed Mar. 6, 2009, which is incorporated herein for all purposes.

However, the problems with such systems is that there is likely to be error in the tracking of each instrument, and those errors will not necessarily cancel each other out. Further, the error can be exacerbated when the instrument is long and when it is flexible. For example, as depicted in FIG. 1, the longer the instrument, the greater the positional estimation error caused by the tracking error. Flexibility in an instrument causes more error the longer the instrument and the more flexible the instrument. All of these errors accumulate and cause a computer imaging system to incorrectly display the relative emplacements (e.g., position and orientation, merely position, or merely orientation) of the tracked instruments and modalities. For example, in an example system of the '899 application, an ultrasound transducer and an ablation needle might both be tracked by an optical tracking system and the doctor may be trying to determine the line in which the ablation needle is pointing in order to target a tumor she has spotted in the ultrasound image. Relative error between the tracking of the ultrasound image and the ablation needle will cause the doctor to incorrectly position and drive the needle. Further, the longer the drive the needle must take in order to reach the plane of the ultrasound image, the more that a small rotational error in the tracking will effect the position of the drive.

These problems and others are addressed by the systems, methods, devices and computer-readable media described herein.

### SUMMARY

Presented herein are methods, systems, devices, and computer-readable media for correction of relative tracking error based on a fiducial. One embodiment is a method for the correction of obtained emplacement data of two surgical instruments by the detection of a detectable fiducial coupled to first surgical instrument by a second surgical instrument or something thereto coupled. The corrected relative emplacements of both surgical instruments is then determined based on the emplacement of the fiducial. This corrected emplacement is then used to produce an image for display. Systems and devices for carrying out the presented methods are also described. Systems, devices, and computer-readable media for carrying out the presented processes are also described herein.

For example, in one embodiment, a process is presented for the correction of obtained emplacement data of two surgical instruments by the detection of a visually-detectable fiducial coupled to first surgical instrument, such as an ablation needle, in an image captured by a second surgical instrument, such as an endoscope or laparoscope. The corrected relative emplacements of both surgical instruments is then determined based on the emplacement of the fiducial in the image obtained. This corrected relative emplacement is then used to produce an image for display. The displayed image may include virtual representations of the ablation needle, its projected ablation volume, and the captured image or video.

### BRIEF DESCRIPTION OF THE DRAWINGS

FIG. 1 illustrates the relative error that can occur for two tracked devices with different length shafts.

FIG. 2 illustrates an exemplary system for presenting corrected medical imaging data.

FIG. 3 illustrates an exemplary surgical instrument marked with two visually-detectable fiducials.

FIG. 4 illustrates the relative correction of emplacements of two surgical instruments.

FIG. 5 is a block diagram that illustrates a method of presenting corrected medical imaging data based on a detectable fiducial.

FIG. 6 illustrates exemplary corrected and uncorrected medical imaging data.

### DETAILED DESCRIPTION OF SPECIFIC EMBODIMENTS

### I. Overview

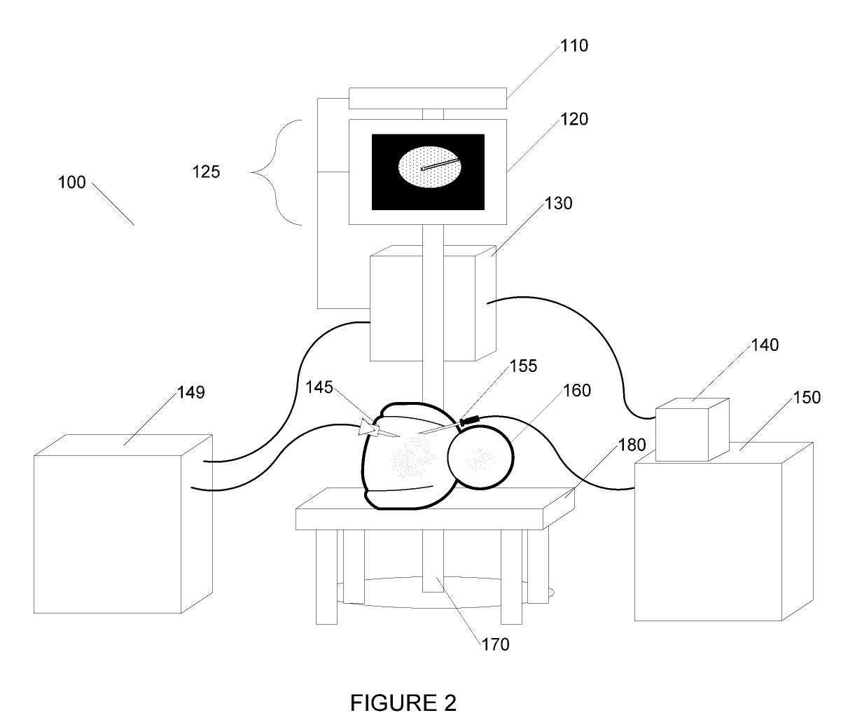

FIG. 2 illustrates an exemplary system for presenting corrected medical imaging data. There are numerous other possible embodiments of system 100, for example, numerous of the depicted modules may be joined together to form a single module and may even be implemented in a single computer or machine. Further, position sensing units 110 and 140 may be combined and track all relevant surgical instruments 145 and 155, as discussed in more detail below. Additional imaging units 150 may be included and combined imaging data from the multiple imaging units 150 may be processed by image guidance unit 130 and shown on display unit 120. Additional surgical devices 149 may also be included. Information about and from multiple surgical devices 149 and attached surgical instruments 145 may be processed by image guidance unit 130 and shown on display 120. These and other possible embodiments are discussed in more detail below.

In some embodiments, image guidance unit 130 takes in imaging information from imaging unit 150. Image guidance unit 130 may attempt to detect a fiducial within an image produced by imaging unit 150. The fiducial may be attached to surgical instrument 145. Image guidance unit 130 may determine the relative emplacements of first surgical instrument 145 and second surgical instrument 155 at least in part based on the placement of the fiducial within the image produced by imaging unit 150. For example, if first surgical instrument 145 is an ablation needle 145 that has a detectable fiducial on it and second surgical instrument 155 is a laparoscopic camera 155, then image guidance unit may correct the tracked emplacements of ablation needle 145 and laparoscopic camera 155 based on where the detectable fiducial attached to the ablation needle is in the image captured by laparoscopic camera 155 and transmitted to image guidance unit 130 from imaging unit 150.

In the pictured embodiment, system 100 comprises a first position sensing unit 110, a display unit 120, and second position sensing unit 140 all coupled to image guidance unit 130. In some embodiments, first position sensing unit 110, display unit 120, and image guidance unit 130 are all physically connected to stand 170. Image guidance unit 130 may be used to produce images 125 that are displayed on display unit 120. The images 125 produced on display unit 120 by the image guidance unit 130 may be made determined based on laparoscopic or other visual images from first surgical instrument 145 and second surgical instrument 155. For example, if first surgical instrument 145 is an ablation needle 145 and second surgical instrument 155 is a laparoscopic camera 155, then images 125 produced on display 120 may include the video from the laparoscopic camera 155 combined with graphics, such as projected ablation volume, determined based on the emplacement of ablation needle 145. Emplacement as used herein may refer to position, orientation, the combination or position and orientation, or any other appropriate location information. In some embodiments, the imaging data obtained from one or both of surgical instruments 145 and 155 may include other modalities such as a CT scan, MRI, open-magnet MRI, optical coherence tomography, positron emission tomography (“PET”) scans, fluoroscopy, ultrasound, or other preoperative or intraoperative anatomical imaging data and any 3 D anatomical imaging data. In some embodiments, surgical instruments 145 and 155 may also be scalpels, implantable hardware, or any other device used in surgery.

As noted above, images 125 produced may also be based on intraoperative or real-time data obtained using second surgical instrument 155, which is coupled to second imaging unit 150. Second surgical instrument 155 may be coupled to second position sensing unit 140. Second position sensing unit 140 may be part of imaging unit 150 or it may be separate. Second position sensing unit 140 may be used to determine the emplacement of second surgical instrument 155. In some embodiments, first and/or second position sensing units 110 and/or 140 may be magnetic trackers and magnetic may be coils coupled to surgical instruments 145 and/or 155. In some embodiments, first and/or second position sensing units 110 and/or 140 may be optical trackers and visually-detectable fiducials may be coupled to surgical instruments 145 and/or 155.

Images 125 produced may also be based on intraoperative or real-time data obtained using first surgical instrument 145, which is coupled to first surgical device 149. In FIG. 2, first surgical device 149 is shown as coupled to image guidance unit 130. The coupling between the first surgical device 149 and image guidance unit 130 may not be present in all embodiments. In some embodiments, the coupling between first surgical device 149 and image guidance unit 130 may be included where information about first surgical instrument 145 available to first surgical device 149 is useful for the processing performed by image guidance unit 130. For example, in some embodiments, first surgical instrument 145 is an ablation needle 145 and first surgical device 149 is an ablation system 149. In some embodiments, it may be useful to send a signal about the relative strength of planned ablation from ablation system 149 to image guidance unit 130 in order that image guidance unit 130 can show a predicted ablation volume. In other embodiments, first surgical device 149 may not be coupled to image guidance unit 130.

In some embodiments, first position sensing unit 110 tracks the emplacement of first surgical device 145. First position sensing unit 110 may be an optical tracker 110 and first surgical device 145 may have optical fiducial attached thereto. The emplacement of optical fiducials may be detected by first position sensing unit 110 and therefrom the emplacement of first surgical device 145 may be determined.

In various embodiments, first position sensing unit 110 and second position sensing unit 140 may be replaced by a single position sensing unit 110 and that single position sensing unit 110 may track both first surgical device 145 and second surgical device 155. In some embodiments, either the first position sensing unit 110 or the second position sensing unit 140 may be an Ascension Flock of Birds, Nest of Birds, driveBAY, medSAFE, trakSTAR, miniBIRD, MotionSTAR, pciBIRD, or Calypso 4D Localization System and tracking units attached to the first and or second surgical devices 145 and 155 may be magnetic tracking coils. In some embodiments, either first position sensing unit 110 or second position sensing unit 140 may be an Aurora® Electromagnetic Measurement System using sensor coils for tracking units attached to the first and or second surgical devices 145 and 155. In some embodiments, first position sensing unit 110 or second position sensing unit 140 may also be an optical 3D tracking system using fiducials. Such optical 3D tracking systems may include the NDI Polaris Spectra, Vicra, Certus, PhaseSpace IMPULSE, Vicon MX, InterSense IS-900, NaturalPoint OptiTrack, Polhemus FastTrak, IsoTrak, or Claron MicronTracker2. In some embodiments, either or both of position sensing units 110 and 140 may be attached to the corresponding surgical device 145 and 155. In these embodiments, the position sensing units, 110 and 140, may include sensing devices such as the HiBall tracking system, a GPS device or signal emitting device that would allow for tracking of the position and, optionally, orientation of the tracking unit. Position sensing devices 145 and 155 may also include one or more accelerometers.

### II. Fiducials and a Surgical Device

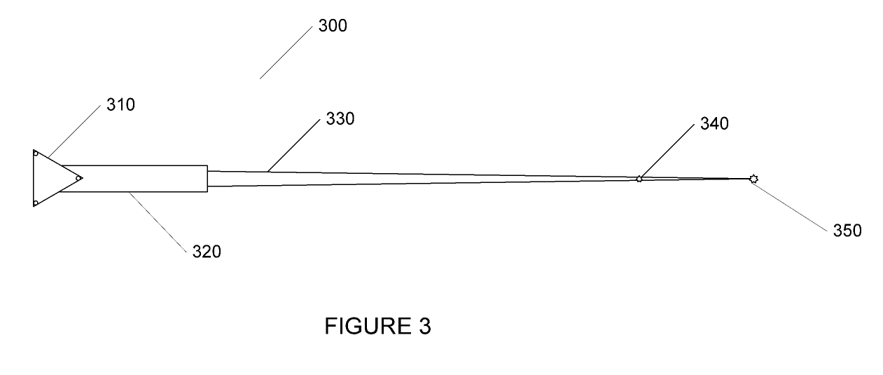

FIG. 3 illustrates an exemplary surgical instrument 300 marked with two detectable fiducials 340 and 350. Surgical instrument 300 may include a trackable portion 310. In some embodiments, the trackable portion of surgical instrument 310 is a set of visual fiducials that are trackable using an optical tracking system, such as a first or second position sensing unit 110 or 140. Trackable portion 310 of the surgical instrument may also be magnetic coils to be used with a magnetic tracker, a GPS or HiBall device, or any corresponding trackable assembly to be used with a position sensing system 110 or 140. In some embodiments, trackable portion 310 may be affixed to or embedded in handle 320 of surgical instrument 300. Trackable portion 310 may also be affixed to or embedded in the portion of the surgical instrument that will be placed within the patient, embeddable portion 330. Trackable portion 310 may be affixed to or embedded in any portion of surgical instrument 300.

The detectable fiducials 340 and 350 may be affixed to or embedded in any portion of surgical instrument 300. Further, there may be any number of fiducials. In an embodiment, there may be two fiducials 340 and 350 affixed to the surgical instrument. In other embodiments, however, there may be one, three, or any number of detectable fiducials 340 and 350 affixed to the instrument. If it is known that the tip of surgical instrument 300 will be embedded within the patient or not detectable, then a fiducial 340 or 350 may be placed in a position, such as further up the shaft of surgical instrument 300 so that it may be visible.

There are various embodiments of detectable fiducials 340 and 350. Further, fiducials 340 and 350 may be different in size, material, and detection method. Detection methods for fiducials are described more below. In some embodiments, fiducials 340 and 350 are visually-detectable. For example, fiducials 340 and 350 may be small, single-colored dots. Complex fiducials, such as bar codes, are more complex and are more difficult to detect and may require more of a surgical instrument's shaft to be visible in, for example, a laparoscopic image.

In some embodiments, a detectable fiducial may be made of a retroreflective material or may be retroreflectors, and the fiducials may reflect light back to a light source with a minimum of scattering. For example, a material made by 3M Corp with a smooth texture (made of microscopic spheres) may be used as a fiducial. In some embodiments, such materials may have the property that the usefulness or symmetry of the retroreflectivity is minimally affected by the presence of blood or other bodily fluids, and the adhesive may continue to adhere inside the body.

In some embodiments, such as those using color laparoscopic images, much of the background is red due to the presence of blood. There may also be many white specular reflections on the tissues and on surgical instrument 300. In some embodiments, a green colored visually-detectable fiducial may allow detection despite the white and red laparoscopic images.

In some embodiments, detectable fiducials 340 and 350 may be visually-detectable fiducials 340 and 350 and in a system, such as system 100 depicted in FIG. 2, may include a surgical instrument such as a laparoscopic or endoscopic camera 155, and image guidance unit 130 may perform optical detection of visually-detectable fiducial 340 or 350 in order to correct the relative emplacements of two surgical instruments 145 and 155. In some embodiments, detectable fiducials 340 and 350 are detectable by other than visual imagery. For example, detectable fiducials 340 and 350 may be emitters, such as radio frequency emitters, or items that reflect other energy forms, such as ultraviolet radiation. In some embodiments, a surgical instrument 155 may be usable to detect the emplacements of detectable fiducials 340 and 350 by detecting the emitted or reflected energy or radiation. For example, if fiducial 340 or 350 were an ultraviolet light emitter and an endoscopic camera 155 could detect emitted ultraviolet light, then image guidance system 130 could use emplacement information of the ultraviolet emitter from endoscopic camera 155 in order to correct the relative emplacements of endoscopic camera 155 and other surgical instrument 145. In some embodiments, the second surgical device 155 may be a 2D or 3D ultrasound wand 155 and the fiducial 340 or 350 may be a vibrating fiducial 340 or 350 or corner cube fiducial 340 or 350. The vibrating fiducial 340 or 350 or corner cube fiducial 340 or 350 may be detectable in the ultrasound image based on the appearance of the vibration or reflection in the ultrasound image. Therefore, the image guidance unit 130 may be able to detect the vibrating fiducial 340 or 350 or corner cube fiducial 340 or 350 from the image generated by the ultrasound wand 155.

### III. Correcting the Relative Emplacements of Two Surgical Instruments

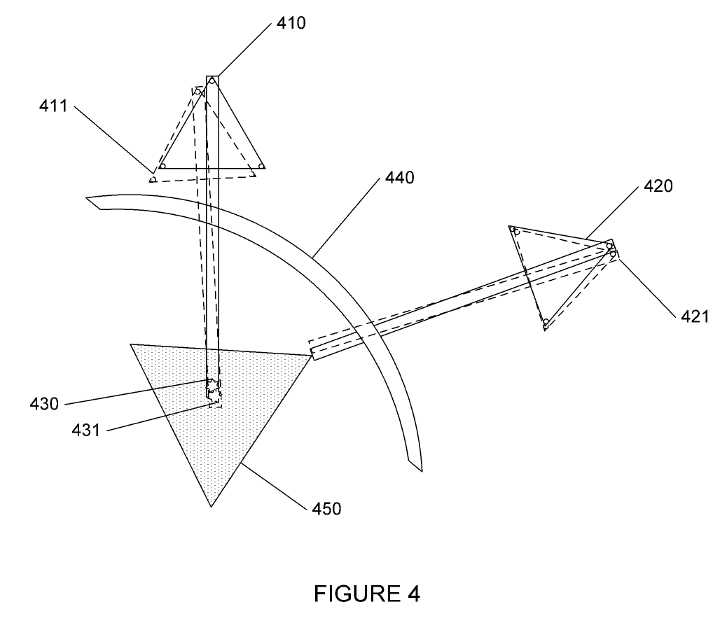

FIG. 4 illustrates the relative correction of emplacements of two surgical instruments 410 and 420. First surgical instrument 410 may be an ablation needle 410. Detectable fiducial 430 may be attached to first surgical instrument 410, which has been inserted through a patient's abdominal wall 440. In some embodiments, also inserted through abdominal wall 440 may be a second surgical instrument 420, such as a laparoscopic camera 420. As noted above, two surgical instruments 410 and 420 may be tracked so that the relative emplacements of two surgical instruments 410 and 420 may be determined. Exemplary embodiments of tracking the surgical instruments are discussed herein.

Laparoscopic camera 420 may have a field of view 450 in which it can capture images of the shaft of ablation needle 410 and attached detectable fiducial 430, such as visual fiducial 430. In some embodiments, based on the location of visual fiducial 430 in the image captured by laparoscopic camera 420, the relative positions of laparoscopic camera 420 and ablation needle 410 may be corrected. Exemplary embodiments of correcting for the location 431 of visual fiducial 430 in the image captured by laparoscopic camera 420 are discussed herein.

Correcting the relative emplacements of two surgical instruments 410 and 420 may comprise determining corrected emplacement 411 of first surgical instrument 410 and/or corrected emplacement 421 of second surgical instrument 420. In some embodiments, when the relative emplacements of surgical instruments 410 and 420 have importance, one may correct the relative emplacement of only one or of both of surgical instruments 410 and 420.

In some embodiments, if there is a higher confidence about the emplacement of one of the surgical instruments 410 or 420, then the emplacements of the other surgical instrument 420 or 410 may be corrected. In some embodiments, first surgical instrument 410 may be made rigid material and second surgical instrument 420 may be made of more flexible material. In this and similar embodiments, the information obtained about the relative emplacements of the two surgical instruments 410 and 420 may be used to correct the emplacement of the second surgical instrument 420.

In some embodiments, first surgical instrument 410 and second surgical instrument 420 may be optically tracked with fiducials affixed to the handles of the surgical instruments 410 and 420. There may be a higher confidence associated with the emplacement data of one of the surgical instruments if a greater number of fiducials used by the optical tracking system are detected on that surgical instrument than on the other surgical instrument. In this and similar embodiments, the emplacement information obtained from the surgical instrument with the higher number of detectable fiducials may be used with a higher confidence to correct the emplacement data of the other surgical instrument.

In some embodiments, first surgical instrument 410 and second surgical instrument 420 may be magnetically tracked with magnetic coils affixed to the handles of the surgical instruments 410 and 420. The emplacement data associated with one of the surgical instruments may have a higher confidence due to electro magnetic interference or distortion detected in the emplacement data associated with the other surgical instrument. In this and similar embodiments, the emplacement information obtained from the instrument with the least amount of magnetic interference or distortion may be used with a higher confidence to correct the emplacement data of the other surgical instrument.

### IV. Presenting Corrected Medical Imaging Data Based on a Detectable Fiducial

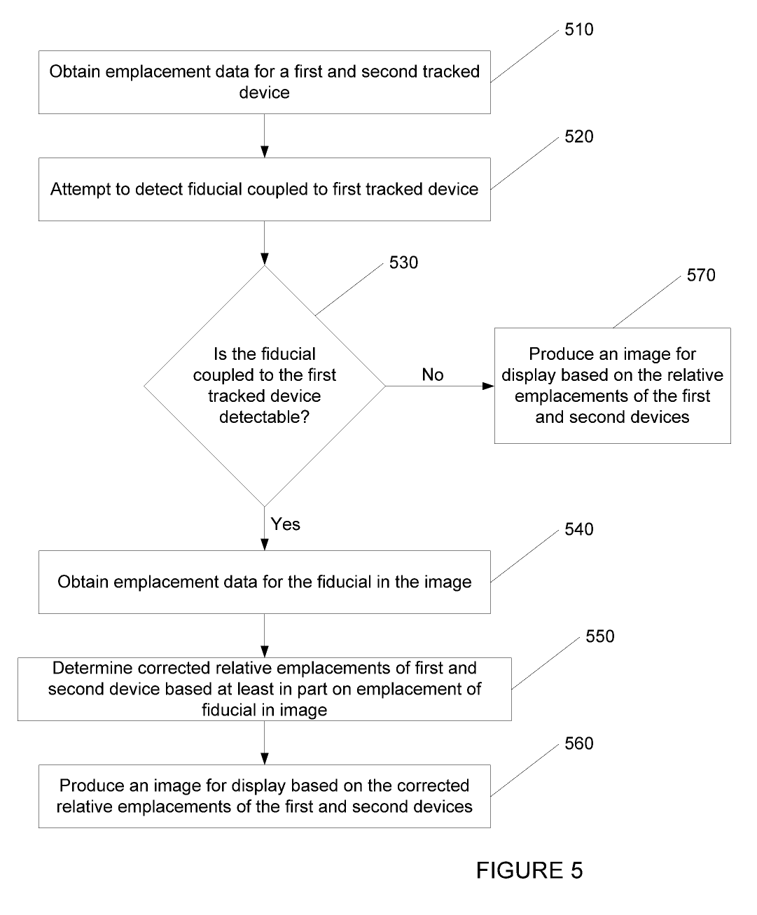

FIG. 5 is a block diagram that illustrates a process for presenting corrected medical imaging data based on a detectable fiducial. In step 510, emplacement data is obtained for two tracked devices. In some embodiments, such as exemplary system 100 in FIG. 2, the first tracked device may be first surgical instrument 145 and the second tracked device may be second surgical instrument 155. In some embodiments, the tracking may be accomplished using first position sending unit 110 and second position sensing unit 140.

In step 520, an attempt is made to detect a fiducial coupled to first tracked device. For example, in some embodiments, first surgical instrument 145, such as ablation needle 145 has thereto attached a visually-detectable fiducial. Second surgical instrument 155, such as laparoscopic camera 155, may take video inside a patient. Image guidance unit 130 may attempt to visually detect the visually-detectable fiducial attached to ablation needle 145 in the image captured by laparoscopic camera 155.

In some embodiments, detecting a fiducial in a red-green-blue (RGB) endoscopic image may be accomplished by the following pseudo code, assuming a green visually-detectable circular fiducial, white illumination, white spectral reflections, and a red background:
1. Subtract the magnitude of the blue component of the RGB image from the magnitude of the green component. In the resultant image, the brightest pixels will be those of the green fiducial.
2. For each row of the image, compute the number of pixels in the row (in the image resulting from step 1) whose value is greater than some fixed threshold. In some embodiments, when using a 24-bit RGB video frame with a camera that has auto-gain/exposure settings, the threshold may be set at or near thirty. In some embodiments, the threshold will depend on the type of camera, illumination, type of image capturing surgical instrument, and fiducial material, including whether it is an emitter or a reflector.
3. For each column of the image, compute the number of pixels in that column whose value is greater than the threshold.
4. The location of the [row, column] center of the fiducial may be the row which has the highest number of above-threshold pixels (from step 2) and the column which has the highest number of above-threshold pixels (from step 3).

In some embodiments, the system may compute the bounding box of a circular region of high-valued pixels as computed in step 1 above, making sure the center position (reported in step 4) is roughly equidistant from the four edges of the bounding box. In some embodiments, this exemplary algorithm, and any of the other algorithms, may be implemented as software for a general purpose central processing unit (CPU), or for a specialized digital signal processor (DSP), field-programmable gate array (FPGA), graphics processor (GPU), or implemented as specialized hardware.

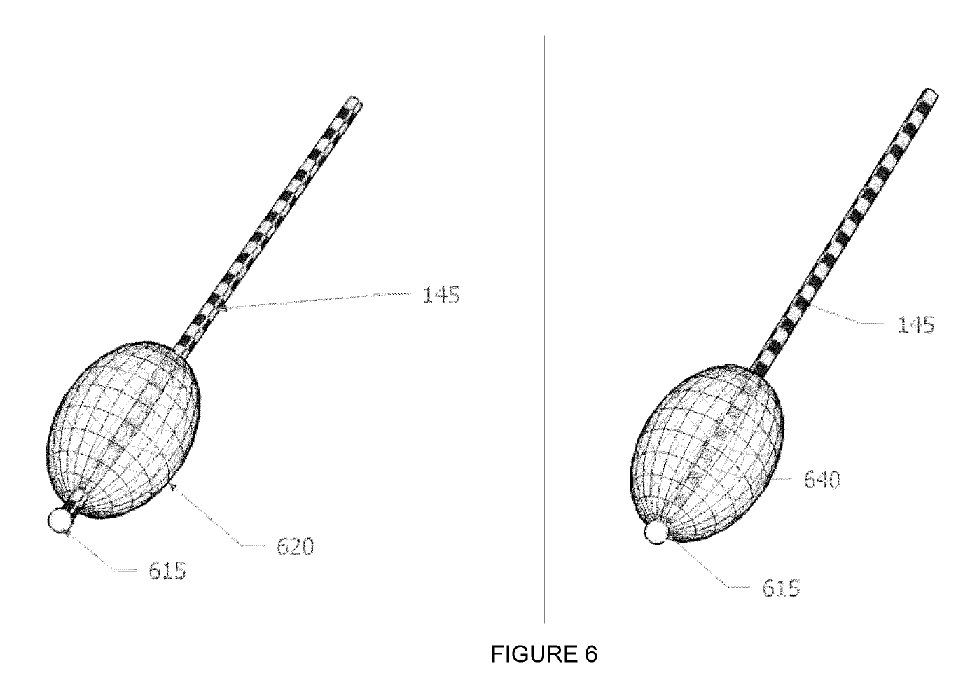

In step 530, if the fiducial is not detected, then an image is produced based on the relative emplacements of the two tracked devices. In some embodiments, continuing with the example above, if a visually-detectable fiducial attached to ablation needle 145 is not detected in the image captured by laparoscopic camera 155, then, in step 570, an image is produced based on the relative emplacements of ablation needle 145 and laparoscopic camera 155. For example, as depicted in FIG. 6 and referencing FIG. 2, if visually-detectable fiducial 615 attached to ablation needle 145 is not detectable in the video image obtained with a laparoscopic camera 155, then image guidance unit 130 may render a projected ablation volume 620. As depicted in FIG. 6, any error in the relative emplacements of laparoscopic camera 155 and ablation needle 145 will result in incorrect placements of the predicted ablation volume 620. If visually-detectable fiducial 615 attached to ablation needle 145 is detectable in the video image obtained with a laparoscopic camera 155, then image guidance unit 130 may render a projected ablation volume 640 based on the corrected relative emplacements of the ablation needle 145 and laparoscopic camera 155, thereby improving the emplacement of the rendered ablation volume 640 relative to the ablation needle 145.

Returning now to FIG. 5, if a fiducial is detected, as determined in step 530, then in step 540, emplacement information for the fiducial is determined. For example, referring to FIG. 2, if first surgical instrument 145 is ablation needle 145 and second surgical instrument 155 is a laparoscopic camera 155, then the placement of visually-detectable fiducial within the image captured by laparoscopic camera 155 may be determined. In some embodiments, if the location of the visually-detectable fiducial within an image is determined, then image guidance unit 130 may be able to correct the relative emplacements of ablation needle 145 and laparoscopic camera 155 by determining where it is predicted that the fiducial should appear in the image captured by laparoscopic camera 155. Image guidance unit 130 may correct the relative emplacements of ablation needle 145 and laparoscopic camera 155 so that the location of the detected fiducial in the image matches the prediction of the location (based on the corrected emplacements of ablation needle 145 and laparoscopic camera 155) of the fiducial. In some embodiments, determining the predicted emplacements of the fiducial in an image captured by second surgical device 155, such as laparoscopic camera 155, will require accounting for the distortion of the camera. This can be accomplished by, for example, rotating the estimate of the emplacement for laparoscopic camera 155 about its center so that the prediction of the location of the fiducial in the image and detection of the location of the fiducial match.

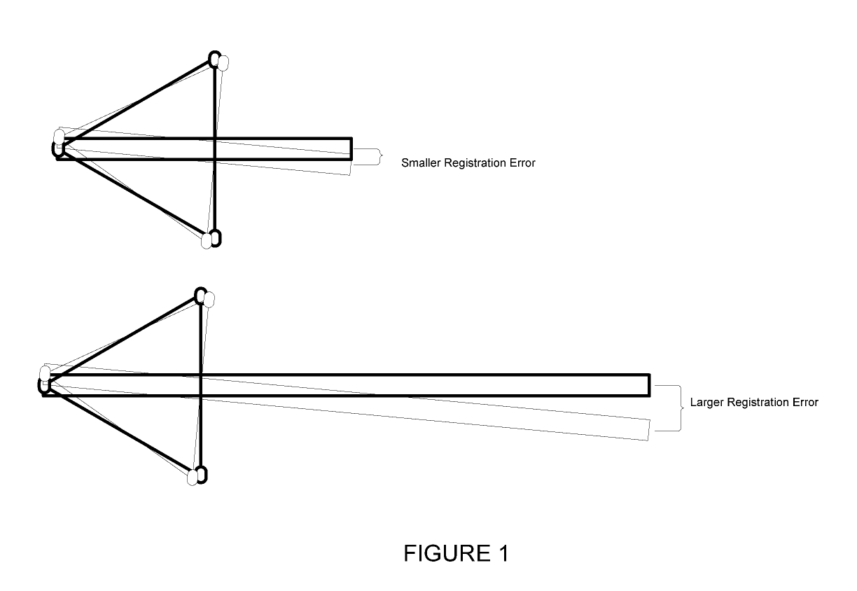

An exemplary pseudo code algorithm for performing steps 530 and 540 may include:
1. First, compute the 3D position (in the camera's coordinate system) of where to expect the center of the fiducial to be, using the reports from the tracking system,
   Fiducial_in_camera=camera_from_endoscopeBody*endoscopeBody_from_tracker*tracker_from_instrument*fiducial_in_instrument
   Where:
   a) fiducial_in_instrument may be the position of the center of the fiducial in the instrument's (such as first surgical instrument 145 in FIG. 2) coordinate system (e.g., in [x,y,z,1] homogenous coordinates).
   b) tracker_from_instrument may be the 4×4 matrix transformation from the instrument's coordinate system to tracker's coordinate system. This, or its inverse, may be reported by the tracking system, such as first position sending unit 110 in FIG. 2.
   c) endoscopeBody_from_tracker may be the 4×4 matrix transformation from trackers's coordinate system (such as the first position sensing unit 140 in FIG. 1, assuming it is tracking the second surgical instrument 155) to the endoscope's (such as second surgical instrument 155) coordinate system. This, or its inverse, may be reported by the tracking system (such as the first position sensing unit 140).
   d) camera_from_endoscopeBody may be the emplacement (e.g., the position and orientation) of the endoscopic camera's optical center (e.i. nodal point) in the endoscope's local coordinate system.
   In some embodiments, the transformations herein may be expressed as a “4×4” or “4 by 4” matrix, or as a positional 3-vector and orientational quaternion, or as a positional 3-vector and orientational Euler angle, or as any other transformation representation. By multiplying all the above matrices, the system may compute the position where the fiducial is expected to appear in the camera's coordinate system.
2. The system may then compute the 2D expected position of the fiducial (x′, y′) in the camera's (such as second surgical instrument 155) image plane using the 3D expected position computed in step 1 (x, y, z, 1), using the formula: x′=x/z y′=y/z.
3. The system may then compute the 2D position of the fiducial's actual location, in the camera's image plane, by taking the detected fiducial location in the video frame, and correcting for the camera's lens distortion. Those having skill in the art will be familiar with how to perform this step. Other embodiments of this step are described herein.
4. The image_plane_offset may be computed as the difference between the expected 2D fiducial position and the determined 2D fiducial position in the captured image.

Correcting the 3D position of the instrument (relative to the camera) may be accomplished by any of numerous possible algorithms, including:
1) The virtual 3D position of the instrument is translated, by the 3d_offset_correction (x″, y″, z″), which is computed by the formula:
   a) x″=−image_plane_offset_x*z
   b) y″=−image_plane_offset_y*z
   c) z″=0
   where z is the z coordinate of the 3-d expected_position of the fiducial in the camera's coordinate system.
2) The virtual camera may be rotated about its nodal point, such that the ray from the nodal point, to the 2D expected fiducial location in the camera's image plane, is (after rotation) co-incident with the ray from the nodal point, to the detected 2D fiducial location in the camera's image plane.

In some embodiments, correcting for the location of a fiducial may entail other transformations or rotations and these may be chosen based on known aspects of the system. For example, if first surgical instrument 145 is attached to an articulating arm and the direction of likely error in tracking the articulated arm is known, then that information could be used to constrain correcting the error. For example, in some embodiments the sections of the arm are rigid, but it is known that there could be an error in determining the angle of one or more of the articulating joints, then the correction of the error of first surgical instrument 145 could be made based, at least in part, based on those constraints (e.g., assuming little translational error in the sections, but attempting to account for error in the prediction of the angle of the joints).

The processes, computer readable medium, and systems described herein may be performed on various types of hardware, such as computer systems. In computer systems may include a bus or other communication mechanism for communicating information, and a processor coupled with the bus for processing information. A computer system may have a main memory, such as a random access memory or other dynamic storage device, coupled to the bus. The main memory may be used to store instructions and temporary variables. The computer system may also include a read-only memory or other static storage device coupled to the bus for storing static information and instructions. The computer system may also be coupled to a display, such as a CRT or LCD monitor. Input devices may also be coupled to the computer system. These input devices may include a mouse, a trackball, or cursor direction keys. Computer systems described herein may include the image guidance unit 130, first and second position sensing units 110 and 140, and imaging unit 150. Each computer system may be implemented using one or more physical computers or computer systems or portions thereof. The instructions executed by the computer system may also be read in from a computer-readable medium. The computer-readable medium may be a CD, DVD, optical or magnetic disk, laserdisc, carrier wave, or any other medium that is readable by the computer system. In some embodiments, hardwired circuitry may be used in place of or in combination with software instructions executed by the processor.

As will be apparent, the features and attributes of the specific embodiments disclosed above may be combined in different ways to form additional embodiments, all of which fall within the scope of the present disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

All of the methods and processes described above may be embodied in, and fully automated via, software code modules executed by one or more general purpose computers or processors, such as those computer systems described above. The code modules may be stored in any type of computer-readable medium or other computer storage device. Some or all of the methods may alternatively be embodied in specialized computer hardware.

It should be emphasized that many variations and modifications may be made to the above-described embodiments, the elements of which are to be understood as being among other acceptable examples. All such modifications and variations are intended to be included herein within the scope of this disclosure and protected by the following claims.

What is claimed is:

1. A method of presenting corrected medical imaging data, comprising:
obtaining emplacement data for a first tracked device;
obtaining emplacement data for a second tracked device;
determining the emplacement of a fiducial coupled to the first tracked device based on information obtained from the second tracked device;
determining corrected relative emplacements of the first and second devices based on the emplacement of the fiducial; and
producing for display an image based on the corrected relative emplacement of the first and second devices.

2. The method of claim 1, where the fiducial is visually detectable.

3. The method of claim 2, where the fiducial is detected in an image.

4. The method of claim 3, where the method for determining the emplacement of the fiducial, comprises:
determining a row in the image with the greatest number of pixels exceeding a threshold;
determining a column in the image with the greatest number of pixels exceeding a threshold; and
determining the intersection of the row and the column.

5. The method of claim 3, where determining the emplacement of the fiducial comprises determining a bounding box for the fiducial in an image captured by the second surgical device.

6. The method of claim 3, wherein determining the corrected relative emplacements of the first and second devices, comprises:
determining an expected position of the fiducial based on the emplacement data of the first and second device;
determining a detected position of the fiducial based in the image;
determining an offset using the expected position and the actual position; and
correcting the position of the first device relative to the second device using the offset.

7. A system that presents corrected medical imaging data, comprising:
a first surgical instrument;
a detectable fiducial coupled to the first surgical instrument;
a second surgical instrument configured to detect the emplacement of the detectable fiducial;
one or more position sensing units for determining the position of the first surgical instrument and the second surgical instrument;
an image guidance unit configured to determine a corrected relative emplacement of the first surgical instrument and second surgical instrument based on the emplacement of the detectable fiducial; and
a display unit configured to display medical imaging data based on the corrected relative emplacements of the first surgical instrument and the second surgical instrument.

8. The system of claim 7, wherein the detectable fiducial is visually-detectable.

9. The system of claim 7, wherein the detectable fiducial is non-visually-detectable.

10. The system of claim 7, wherein the first surgical instrument is tracked by a first position sensing unit and the second surgical instrument is tracked by a second position sensing unit.

11. The system of claim 7, wherein one or more position sensing units comprise one or more trackers selected from the group consisting of a magnetic tracker and an optical tracker.

12. The system of claim 7, wherein the first instrument is an ablation needle.

13. The system of claim 7, wherein the second surgical instrument comprises a camera.

14. The system of claim 7, wherein the second surgical instrument is an ultrasound wand and the detectable fiducial is selected from the group consisting of a vibrating fiducial and a corner cube fiducial.

15. A device, comprising:
a surgical instrument;
at least one visually-detectable fiducial coupled to the surgical instrument that is trackable using an optical tracking system; and
a trackable portion that is trackable by a positioning system.

16. The device of claim 15, wherein the detectable fiducial is single-colored.

17. The device of claim 15, wherein the detectable fiducial is made of retroreflective material.

18. The device of claim 15, wherein the trackable portion is electro-magnetically tracked.

19. The device of claim 15, wherein the trackable portion is optically tracked.

20. A system that presents corrected imaging data, comprising:
a first instrument;
a detectable fiducial coupled to the first instrument;
a second instrument configured to detect the emplacement of the detectable fiducial;
one or more position sensing units for determining the position of the first instrument and the second instrument;
an image guidance unit configured to determine a corrected relative emplacement of the first instrument and second instrument based on the emplacement of the detectable fiducial; and
a display unit configured to display imaging data based on the corrected relative emplacements of the first instrument and the second instrument.
